(19) United States
(12) Patent Application Publication
(10) Pub. No.: US 2025/0209837 A1
(43) Pub. Date: Jun. 26, 2025

(54) ULTRASOUND TOMOGRAPHIC IMAGE PROCESSING APPARATUS AND ULTRASOUND TOMOGRAPHIC IMAGE PROCESSING PROGRAM

(71) Applicant: FUJIFILM Corporation, Tokyo (JP)

(72) Inventors: Atsushi SHIROMARU, Tokyo (JP); Tsuyoshi MATSUMOTO, Tokyo (JP)

(73) Assignee: FUJIFILM Corporation, Tokyo (JP)

(21) Appl. No.: 18/979,661

(22) Filed: Dec. 13, 2024

(30) Foreign Application Priority Data
Dec. 20, 2023 (JP) 2023-214773

(51) Int. Cl. G06V 20/69; G06T 7/00; G16H 30/20; G16H 30/40

(52) U.S. Cl. CPC G06V 20/695; G06T 7/0012; G16H 30/20; G16H 30/40; G06T 2207/10132; G06T 2207/30168

(57) ABSTRACT

An ultrasound tomographic image processing apparatus including a cross section type specification unit, a tissue structure specification unit, and a display controller is provided. The cross section type specification unit inputs a target image to a first learning model and specifies a cross section type of the target image on the basis of a prediction result of the first learning model for the target image. The tissue structure specification unit inputs the target image to an initial second learning model associated with a specific cross section to specify a first tissue structure included in the target image. The display controller displays first tissue structure information related to the first tissue structure on a display. The tissue structure specification unit inputs the target image to other second learning models other than the initial second learning model in response to an instruction from a user and specifies a second tissue structure included in the target image.

### CROSS-REFERENCE TO RELATED APPLICATION

This application claims priority under 35 USC 119 from Japanese Patent Application No. 2023-214773, filed 20 Dec. 2023, the disclosure of which is incorporated by reference herein.

### BACKGROUND OF THE INVENTION

### 1. Field of the Invention

The present specification discloses improvements in an ultrasound tomographic image processing apparatus and an ultrasound tomographic image processing program.

### 2. Description of the Related Art

In the related art, there is a technique that analyzes an ultrasound tomographic image (B-mode image) formed by an ultrasound diagnostic apparatus to specify a tissue structure (for example, an organ, a blood vessel, or the like) included in the ultrasound tomographic image. In the related art, an ultrasound diagnostic apparatus having this function is also present. For example, the specification of the tissue structure in the ultrasound tomographic image makes it possible to perform measurement or the like related to the specified tissue structure using the ultrasound tomographic image.

For example, JP6836652B discloses an ultrasound diagnostic apparatus that performs an image recognition process, such as a process using a learning model or a pattern matching process with template data, on a formed ultrasound tomographic image to automatically discriminate a tissue structure as an object to be measured in the ultrasound tomographic image.

### SUMMARY OF THE INVENTION

However, it is considered to specify a tissue structure included in an ultrasound tomographic image, using a first learning model that predicts a cross section type of the ultrasound tomographic image and outputs the cross section type and a plurality of second learning models that predict the tissue structure included in the ultrasound tomographic image and output the tissue structure. Each of the plurality of second learning models is associated with each cross section type. In this case, first, an ultrasound tomographic image to be processed is input to the first learning model, and the cross section type of the ultrasound tomographic image is specified by the first learning model. Then, the ultrasound tomographic image is input to the second learning model associated with the specified cross section type, and the tissue structure included in the ultrasound tomographic image is specified by the second learning model.

In a case where the tissue structure is specified by the above-described process, it is desired to improve the accuracy of specifying the tissue structure.

An object of an ultrasound tomographic image processing apparatus according to a present embodiment is to improve accuracy of specification in a case of specifying a tissue structure included in an ultrasound tomographic image using a first learning model that predicts a cross section type of the ultrasound tomographic image and a second learning model that predicts a tissue structure included in an ultrasound tomographic image of a specific cross section type.

The present specification discloses an ultrasound tomographic image processing apparatus capable of accessing a first learning model that has been trained to predict a cross section type of an input ultrasound tomographic image and to output the cross section type and a plurality of second learning models each of which is associated with each cross section type of ultrasound tomographic images and has been trained to predict a tissue structure included in the ultrasound tomographic image of a corresponding cross section type and to output the tissue structure. The ultrasound tomographic image processing apparatus comprises: a cross section type specification unit that inputs a target image, which is an ultrasound tomographic image to be processed, to the first learning model to specify a specific cross section which is a cross section type of the target image; a tissue structure specification unit that inputs the target image to an initial second learning model, which is the second learning model associated with the specific cross section, to specify a first tissue structure included in the target image; and a display controller that displays first tissue structure information, which is information related to the first tissue structure, on a display unit. The tissue structure specification unit inputs the target image to the second learning models, which are other than the initial second learning model and include the second learning model associated with a cross section type other than the specific cross section, in response to an instruction indicating a position on the target image from a user who has checked the first tissue structure information and specifies a second tissue structure included in a vicinity of the position indicated by the instruction from the user in the target image on the basis of prediction results of the second learning models.

The display controller may display cross section information indicating the specific cross section on the display unit. In a case where the specific cross section is different from a cross section type associated with the second learning model that has contributed to the specification of the second tissue structure, the display controller may display corrected cross section information indicating the cross section type corresponding to the second learning model that has contributed to the specification of the second tissue structure on the display unit, instead of the cross section information indicating the specific cross section.

The plurality of second learning models may be grouped corresponding to each part of a subject, and the tissue structure specification unit may input the target image to the second learning model belonging to the same group as the initial second learning model among the plurality of second learning models other than the initial second learning model in response to an instruction from the user who has checked the first tissue structure information and may further specify the second tissue structure included in the target image on the basis of a prediction result of the second learning model.

The tissue structure specification unit may input the target image to each of a plurality of the second learning models other than the initial second learning model in response to an instruction from the user who has checked the first tissue structure information and may calculate an order of prediction accuracy for each of labels of a plurality of tissue structures predicted by the plurality of second learning models. The display controller may display the labels of the plurality of tissue structures predicted by the plurality of second learning models on the display unit in a display mode in which the order of the prediction accuracy is represented, and the tissue structure specification unit may specify, as the second tissue structure, a tissue structure related to a label selected by the user among the plurality of tissue structures predicted by the plurality of second learning models.

The ultrasound tomographic image processing apparatus may further comprise an image quality adjustment unit that adjusts quality of the ultrasound tomographic image on the basis of image quality adjustment information in which the cross section type of the ultrasound tomographic image is associated with an image quality adjustment parameter for adjusting the quality of the ultrasound tomographic image of the cross section type and that, in a case where the specific cross section is different from the cross section type associated with the second learning model which has contributed to the specification of the second tissue structure, adjusts quality of the target image using the image quality adjustment parameter associated with the cross section type corresponding to the second learning model which has contributed to the specification of the second tissue structure.

In addition, the present specification discloses an ultrasound tomographic image processing program causing a computer, which is capable of accessing a first learning model that has been trained to predict a cross section type of an input ultrasound tomographic image and to output the cross section type and a plurality of second learning models each of which is associated with each cross section type of ultrasound tomographic images and has been trained to predict a tissue structure included in an ultrasound tomographic image of a corresponding cross section type and to output the tissue structure, to function as: a cross section type specification unit that inputs a target image, which is an ultrasound tomographic image to be processed, to the first learning model to specify a specific cross section which is a cross section type of the target image; a tissue structure specification unit that inputs the target image to an initial second learning model, which is the second learning model associated with the specific cross section, to specify a first tissue structure included in the target image; and a display controller that displays first tissue structure information, which is information related to the first tissue structure, on a display unit. The tissue structure specification unit inputs the target image to the second learning models, which are other than the initial second learning model and include the second learning model associated with a cross section type other than the specific cross section, in response to an instruction indicating a position on the target image from a user who has checked the first tissue structure information and specifies a second tissue structure included in a vicinity of the position indicated by the instruction from the user in the target image on the basis of prediction results of the second learning models.

According to the ultrasound tomographic image processing apparatus disclosed in the present specification, it is possible to improve the accuracy of specification in a case of specifying the tissue structure included in the ultrasound tomographic image, using the first learning model that predicts the cross section type of the ultrasound tomographic image and the second learning model that predicts the tissue structure included in the ultrasound tomographic image of the specific cross section type.

### BRIEF DESCRIPTION OF THE DRAWINGS

FIG. 1 is a schematic diagram illustrating a configuration of an ultrasound diagnostic apparatus according to a present embodiment.

FIG. 2 is a diagram illustrating a display example of first tissue structure information.

FIG. 3 is a diagram illustrating a display example of second tissue structure information.

FIG. 4 is a diagram illustrating a display example of corrected cross section information.

FIG. 5 is a diagram illustrating a display example of labels of a plurality of tissue structures.

FIG. 6 is a flowchart illustrating a flow of a process of the ultrasound diagnostic apparatus according to the present embodiment.

### DESCRIPTION OF THE PREFERRED EMBODIMENTS

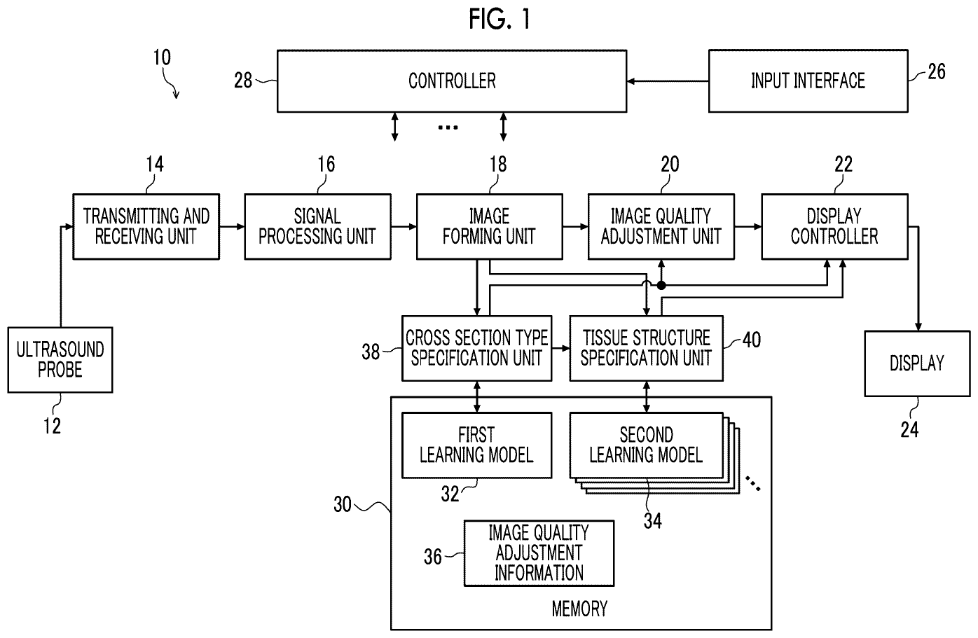

FIG. 1 is a schematic diagram illustrating a configuration of an ultrasound diagnostic apparatus 10 as an ultrasound tomographic image processing apparatus according to a present embodiment. The ultrasound diagnostic apparatus 10 is a medical apparatus that is installed in a medical institution such as a hospital.

The ultrasound diagnostic apparatus 10 is an apparatus that scans a subject with an ultrasound beam and generates an ultrasound image, which is a medical image, on the basis of a received signal obtained by the scanning. In particular, in the present embodiment, the ultrasound diagnostic apparatus 10 forms an ultrasound tomographic image (B-mode image) obtained by converting amplitude strength of reflected waves from a scanning surface into brightness, on the basis of the received signal. In addition, the ultrasound diagnostic apparatus 10 can also form other ultrasound images, such as Doppler images that are formed on the basis of a difference (Doppler shift) in frequency between transmission waves and received waves and that represent a motion velocity of a tissue in the subject.

The ultrasound diagnostic apparatus 10 performs a process of analyzing the formed ultrasound tomographic image to specify a tissue structure in the subject, which will be described in detail below. In the present embodiment, the ultrasound diagnostic apparatus 10 performs a process, such as measurement, on the specified tissue structure. That is, in the present embodiment, the tissue structure to be specified is an object to be measured.

In addition, a transmitting and receiving unit 14, a signal processing unit 16, an image forming unit 18, an image quality adjustment unit 20, a display controller 22, a cross section type specification unit 38, and a tissue structure specification unit 40 included in the ultrasound diagnostic apparatus 10 are configured by a processor. The processor is configured to include at least one of a general-purpose processing device (for example, a central processing unit (CPU)) or a dedicated processing device (for example, a graphics processing unit (GPU), an application specific integrated circuit (ASIC), a field programmable gate array (FPGA), or a programmable logic device). The processor may be configured by a cooperation between a plurality of processing devices that are present at physically separated positions, instead of being configured by one processing device. In addition, each of the above-described units may be implemented by a cooperation between hardware, such as a processor, and software.

An ultrasound probe 12 is a device that transmits and receives ultrasound waves to and from the subject (particularly, the tissue structure to be measured). The ultrasound probe 12 has a transducer array including a plurality of transducers that transmit and receive the ultrasound waves to and from the subject.

The transmitting and receiving unit 14 transmits a transmission signal to the ultrasound probe 12 (specifically, each transducer of the transducer array) under the control of a controller 28 (which will be described below). Then, the ultrasound waves are transmitted from each transducer toward the subject.

In addition, the transmitting and receiving unit 14 receives a received signal from each transducer that has received the reflected waves from the subject. The transmitting and receiving unit 14 includes an adder and a plurality of delayers corresponding to each transducer, and a phase matching and addition process of matching and adding phases of the received signals from each of the transducers is performed by the adder and the plurality of delayers. Therefore, a received beam signal in which information indicating the signal intensity of the reflected waves from the subject is arranged in a depth direction of the subject is formed.

The signal processing unit 16 executes various types of signal processing including a filtering process of applying a bandpass filter, a detection process, and the like on the received beam signal from the transmitting and receiving unit 14.

The image forming unit 18 forms an ultrasound tomographic image (B-mode image) on the basis of the received beam signal subjected to the signal processing by the signal processing unit 16. First, the image forming unit 18 converts the received beam signal into data in a coordinate space of the ultrasound image. Then, the image forming unit 18 forms the ultrasound tomographic image on the basis of the coordinate-converted signal.

The image quality adjustment unit 20 executes a process of adjusting the quality (for example, brightness) of the ultrasound tomographic image formed by the image forming unit 18. The image quality adjustment unit 20 executes an image quality adjustment process corresponding to a processing result (that is, the cross section type of the ultrasound tomographic image) of the cross section type specification unit 38 which will be described below. The processing content of the image quality adjustment unit 20 will be described in detail below.

The display controller 22 performs control to display, on a display 24, the ultrasound tomographic image formed by the image forming unit 18 and various other types of information. The display 24 as a display unit is, for example, a display device configured by a liquid crystal display, an organic electro luminescence (EL), or the like.

An input interface 26 is configured by, for example, buttons, a track ball, a touch panel, or the like. The input interface 26 is used to input a command from a user to the ultrasound diagnostic apparatus 10. In the present embodiment, the display 24 is a touch panel and also functions as the input interface 26.

The controller 28 is configured to include at least one of a general-purpose processor (for example, a central processing unit (CPU)) or a dedicated processor (for example, a graphics processing unit (GPU), an application specific integrated circuit (ASIC), a field programmable gate array (FPGA), a programmable logic device, or the like). The controller 28 may be configured by a cooperation between a plurality of processing devices that are present at physically separated positions, instead of being configured by one processing device. The controller 28 controls each unit of the ultrasound diagnostic apparatus 10 according to an ultrasound tomographic image processing program stored in a memory 30 which will be described below.

The memory 30 is configured to include a hard disk drive (HDD), a solid state drive (SSD), an embedded multi media card (eMMC), a read only memory (ROM), or the like. The memory 30 is connected to each of the units so as to be accessible from the processor of the ultrasound diagnostic apparatus 10, specifically, each of the units illustrated in FIG. 1 which include the controller 28, and the cross section type specification unit 38 and the tissue structure specification unit 40 which will be described below. The memory 30 stores the ultrasound tomographic image processing program for operating each unit of the ultrasound diagnostic apparatus 10. In addition, the ultrasound tomographic image processing program can also be stored, for example, in a non-transitory computer-readable storage medium such as a universal serial bus (USB) memory or a CD-ROM. The ultrasound diagnostic apparatus 10 can read the ultrasound tomographic image processing program from the storage medium and execute the ultrasound tomographic image processing program.

Further, as illustrated in FIG. 1, a first learning model 32 and a plurality of second learning models 34 are stored in the memory 30.

The first learning model 32 is configured as, for example, a model, such as a convolutional neural network (CNN), but may be any model as long as it can exhibit functions which will be described below. The first learning model 32 uses the ultrasound tomographic image as input data, predicts the cross section type of the input ultrasound tomographic image, and outputs the prediction result of the cross section type.

In the present embodiment, the first learning model 32 is trained by a learning device, which is a device other than the ultrasound diagnostic apparatus 10, and the trained first learning model 32 is stored in the memory 30. A processor of the learning device trains the first learning model 32 using a combination of the ultrasound tomographic image and information (training data) indicating the cross section type of the ultrasound tomographic image as learning data. Specifically, the processor of the learning device inputs the ultrasound tomographic image, which is the learning data, to the first learning model 32. The first learning model 32 predicts the cross section type of the input ultrasound tomographic image and outputs the prediction result of the cross section type. The processor of the learning device adjusts parameters of the first learning model 32 such that a difference between the cross section type predicted by the first learning model 32 and the cross section type, which is the training data, is reduced. The repetition of the learning process makes it possible for the trained first learning model 32 to predict the cross section type of the ultrasound tomographic image and to output the prediction result. However, even in a case where the first learning model 32 has been sufficiently trained, the first learning model 32 may not completely accurately predict the cross section type of the ultrasound tomographic image and output an inaccurate prediction result.

One first learning model 32 may be stored in the memory 30, or a plurality of first learning models 32 corresponding to a plurality of parts (for example, an abdomen, a lower limb, and the like) of the subject may be stored in the memory 30. In this case, each first learning model 32 is a model that specializes in predicting the cross section type of the ultrasound tomographic image of the corresponding part. For example, the first learning model 32 corresponding to the abdomen is a model specializing in predicting the cross section type of the ultrasound tomographic image formed by transmitting and receiving the ultrasound waves to and from the abdomen, and the first learning model 32 corresponding to the lower limb is a model specializing in predicting the cross section type of the ultrasound tomographic image formed by transmitting and receiving the ultrasound waves to and from the lower limb. In this case, each first learning model 32 is trained using the ultrasound tomographic image of the corresponding part as the learning data. For example, the first learning model 32 corresponding to the abdomen is trained, using the ultrasound tomographic image formed by transmitting and receiving the ultrasound waves to and from the abdomen as the learning data, and the first learning model 32 corresponding to the lower limb is trained, using the ultrasound tomographic image formed by transmitting and receiving the ultrasound waves to and from the lower limb as the learning data.

The second learning model 34 is also configured as a model, such as a CNN, but may be any model as long as it can exhibit functions which will be described below. The second learning model 34 uses the ultrasound tomographic image as input data, predicts the tissue structure included in the input ultrasound tomographic image, and outputs the prediction result of the tissue structure. In the present specification, the prediction (or specification) of the tissue structure included in the ultrasound tomographic image means the prediction (or specification) of at least one of the position of the tissue structure on the ultrasound tomographic image (hereinafter, simply referred to as a “position of a tissue structure”) or a label of the tissue structure (the name of the tissue structure). The second learning model 34 according to the present embodiment predicts the position and label of the tissue structure.

In the present embodiment, the second learning model 34 is trained by a learning device, which is a device other than the ultrasound diagnostic apparatus 10, and the trained second learning model 34 is stored in the memory 30. A processor of the learning device trains the second learning model 34 using, as learning data, a combination of the ultrasound tomographic image and information (training data) indicating the position and label of the tissue structure included in the ultrasound tomographic image. Specifically, the processor of the learning device inputs the ultrasound tomographic image, which is the learning data, to the second learning model 34. The second learning model 34 predicts the position and label of the tissue structure included in the input ultrasound tomographic image and outputs the prediction results of the position and label. The processor of the learning device adjusts parameters of the second learning model 34 such that a difference between the position of the tissue structure predicted by the second learning model 34 and the position of the tissue structure, which is the training data, is reduced. In addition, the processor of the learning device adjusts the parameter of the second learning model 34 such that a difference between the label of the tissue structure predicted by the second learning model 34 and the label of the tissue structure, which is the training data, is reduced. The repetition of the learning process makes it possible for the trained second learning model 34 to predict the position and label of the tissue structure included in the ultrasound tomographic image and to output the prediction results. Further, in a case where the second learning model 34 predicts one of the position or the label of the tissue structure, one of the position or the label of the tissue structure may be included as the training data included in the learning data.

A plurality of second learning models 34 are stored in the memory 30. The plurality of second learning models 34 are associated with the cross section types of the ultrasound tomographic images, respectively. For example, a certain second learning model 34 is associated with a sapheno femoral junction (SFJ) cross section, and another second learning model 34 is associated with a common femoral vein (CFV) cross section. Each of the second learning models 34 is a model that specializes in predicting the tissue structure included in the ultrasound tomographic image of the corresponding cross section type. For example, the second learning model 34 associated with the SFJ cross section is a model specializing in predicting the tissue structure included in the ultrasound tomographic image whose cross section type is the SFJ cross section, and the second learning model 34 associated with the CFV cross section is a model specializing in predicting the tissue structure included in the ultrasound tomographic image whose cross section type is the CFV cross section. Each of the second learning models 34 is trained using the ultrasound tomographic image of the corresponding cross section type as the learning data. For example, the second learning model 34 corresponding to the SFJ cross section is trained using the ultrasound tomographic image, whose cross section type is the SFJ cross section, as the learning data, and the second learning model 34 corresponding to the CFV cross section is trained using the ultrasound tomographic image, whose cross section type is the CFV cross section, as the learning data.

One second learning model 34 may be associated with one cross section type of the ultrasound tomographic image. However, in the present embodiment, a plurality of different second learning models 34 are associated with one cross section type. For example, a plurality of different second learning models 34 are associated with the SFJ cross section. A plurality of second learning models 34 associated with one cross section type have different parameters (including parameters adjusted by the learning process or hyperparameters that are not adjusted by the learning process) or have different contents of pre-processing (which processes input data prior to a prediction process by the learning model). That is, in a case where the same ultrasound tomographic image is input, the plurality of second learning models 34 associated with one cross section type may have different prediction results for the ultrasound tomographic image.

Further, in the present embodiment, the first learning model 32 and the second learning model 34 are trained by a learning device different from the ultrasound diagnostic apparatus 10. However, the first learning model 32 and the second learning model 34 may be trained by the ultrasound diagnostic apparatus 10. In this case, the ultrasound diagnostic apparatus 10 has a learning processing unit (not illustrated in FIG. 1) that is configured by a processor or is implemented by the cooperation between hardware, such as a processor, and software. The learning processing unit executes a process of training the first learning model 32 and the second learning model 34.

Further, as illustrated in FIG. 1, image quality adjustment information 36 is stored in the memory 30. The image quality adjustment information 36 will be described in detail below.

The cross section type specification unit 38 specifies the cross section type of the ultrasound tomographic image formed by the image forming unit 18. In the present specification, the ultrasound tomographic image to be processed by the cross section type specification unit 38 (and the tissue structure specification unit 40) is referred to as a target image.

Specifically, the cross section type specification unit 38 inputs the target image to the first learning model 32 and specifies the cross section type of the target image on the basis of the prediction result of the first learning model 32 for the target image. In the present specification, the cross section type of the target image specified by the cross section type specification unit 38 is referred to as a specific cross section.

The tissue structure specification unit 40 specifies the tissue structure included in the target image. First, the tissue structure specification unit 40 inputs the target image to the second learning model 34 associated with the specific cross section specified by the cross section type specification unit 38 among the plurality of second learning models 34 stored in the memory 30 and specifies the tissue structure included in the target image on the basis of the prediction result of the second learning model 34 for the target image. In a case where a plurality of second learning models 34 are associated with the specific cross section, the tissue structure specification unit 40 inputs the target image to a predetermined second learning model 34 among the plurality of second learning models 34. In the present specification, as described above, one second learning model 34 selected according to the specific cross section specified by the cross section type specification unit 38 is referred to as an initial second learning model 34. In addition, the tissue structure specified by the tissue structure specification unit 40 on the basis of the output of the initial second learning model 34 is referred to as a first tissue structure.

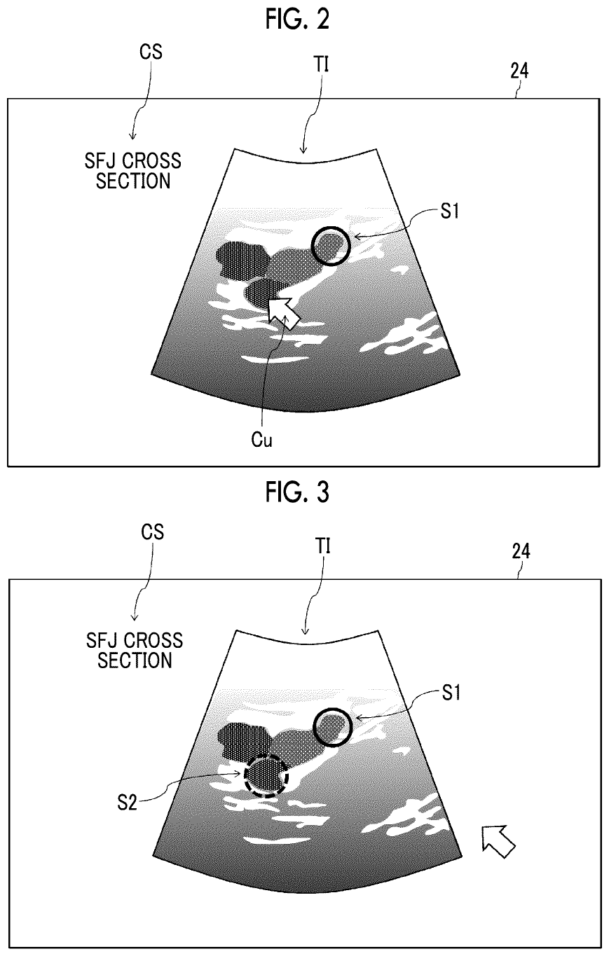

The display controller 22 displays first tissue structure information, which is information related to the first tissue structure specified by the tissue structure specification unit 40, on the display 24. FIG. 2 is a diagram illustrating a display example of first tissue structure information S1. In the present embodiment, the display controller 22 displays a target image TI, cross section information CS indicating the specific cross section, and the first tissue structure information S1 on the display 24. The first tissue structure information S1 includes, for example, an icon indicating the position of the first tissue structure on the target image TI, or an image icon or characters indicating the label. In the example illustrated in FIG. 2, as the first tissue structure information S1, an icon indicating the position of the first tissue structure on the target image TI is displayed to be superimposed on the target image TI. In addition, for example, characters indicating the label of the first tissue structure may be displayed.

Here, it is assumed that the first tissue structure is incorrect. The first tissue structure being incorrect includes a case where the position and label of the specified first tissue structure are matched with the fact, but the specified first tissue structure is different from the tissue structure expected by the user. For example, there is a case where the femoral vein is certainly present at the position indicated by the first tissue structure information S1 and is also labeled as the femoral vein, but the user wants to specify the position of the femoral artery. In addition, the first tissue structure being incorrect also includes a case where the position or label of the specified first tissue structure is different from the fact. For example, there is a case where the femoral vein is correctly present at the position indicated by the first tissue structure information S1, but is labeled as the femoral artery. In the present specification, it is assumed that, in a case where the user wants to specify the position of the femoral artery, the position and label of the femoral vein are specified as the first tissue structure. The position indicated by the first tissue structure information S1 in FIG. 2 is the position of the femoral vein.

A first factor that causes the tissue structure specification unit 40 to specify an incorrect first tissue structure is that the specific cross section specified by the cross section type specification unit 38 is incorrect. As described above, the tissue structure specification unit 40 specifies the first tissue structure using the second learning model 34 associated with the specific cross section, that is, the second learning model 34 specializing in predicting the tissue structure included in the ultrasound tomographic image of the specific cross section. Here, in a case where the cross section type specification unit 38 specifies a cross section type different from the true cross section type of the target image TI as the specific cross section, the tissue structure specification unit 40 specifies the first tissue structure using the second learning model 34 that does not specialize in the true cross section type of the target image TI. For example, in a case where the true cross section type of the target image TI is the CFV cross section, but the cross section type specification unit 38 specifies the SFJ cross section as the specific cross section, the tissue structure specification unit 40 specifies the first tissue structure from the target image TI of the CFV cross section, using the second learning model 34 specializing in the SFJ cross section. In this case, an incorrect first tissue structure can be specified.

A second factor that causes the tissue structure specification unit 40 to specify the incorrect first tissue structure is that the specific cross section specified by the cross section type specification unit 38 is correct, but the initial second learning model 34 is not suitable for the target image TI. As described above, in a case where a plurality of second learning models 34 are associated with the specific cross section, the tissue structure specification unit 40 sets one of the plurality of second learning models 34 as the initial second learning model 34. This is a case where the initial second learning model 34 selected in this way is not suitable for the target image TI. For example, in a case where the brightness of the target image TI is too low for the initial second learning model 34 to predict the correct tissue structure, the incorrect first tissue structure can be specified.

The user checks the first tissue structure information S1 displayed on the display 24 to check that the first tissue structure is incorrect. The user who has checked the error in the first tissue structure inputs an instruction indicating the position of a target tissue structure on the target image TI from the input interface 26. The instruction is also an instruction to notify the tissue structure specification unit 40 that the first tissue structure is incorrect, together with the position of the tissue structure expected by the user on the target image TI. In the present embodiment, the user places a cursor C, which is moved on the display 24 in response to an operation of the input interface 26, at a target position and clicks the position to input the instruction. Here, it is assumed that the user inputs an instruction indicating the position of the femoral artery which is the position of the cursor C illustrated in FIG. 2.

In response to the instruction from the user, the tissue structure specification unit 40 inputs the target image TI to one or a plurality of second learning models 34 that are other than the initial second learning model 34 and include the second learning model 34 associated with the cross section type other than the specific cross section specified by the cross section type specification unit 38. In the present specification, one or a plurality of second learning models 34 which are other than the initial second learning model 34 and to which the target image TI is input are referred to as other second learning models 34. The tissue structure specification unit 40 specifies the tissue structure included in the vicinity of the position indicated by the instruction from the user in the target image TI on the basis of the prediction results of other second learning models 34. In the present specification, the tissue structure specified by the tissue structure specification unit 40 on the basis of the outputs of other second learning models 34 is referred to as a second tissue structure.

Specifically, the tissue structure specification unit 40 sets, as the second tissue structure, a tissue structure detected at a position closest to the position designated by the user among the tissue structures predicted by one or a plurality of other second learning models 34. Alternatively, the tissue structure specification unit 40 may determine a predetermined range having the position designated by the user as the center and may set a tissue structure detected within the range as the second tissue structure. In this configuration, in a case where a plurality of tissue structures are detected within the range by a plurality of other second learning models 34, a tissue structure having the most frequently detected label among the labels of the tissue structures may be set as the second tissue structure. For example, in a case where three other second learning models 34 detect tissue structures having a label “femoral artery” and one other second learning model 34 detects a tissue structure having a label “thrombus” within the range, the tissue structure specification unit 40 sets one of the tissue structures having the label “femoral artery” as the second tissue structure.

In addition, the tissue structure specification unit 40 may cut out a predetermined range having the position designated by the user as the center from the target image TI and input the cut-out image to other second learning models 34 to specify the second tissue structure.

The specified second tissue structure can be a correct tissue structure (that is, the position and label of the first tissue structure are matched with the fact, and the first tissue structure is the tissue structure expected by the user).

First, a case will be considered where the tissue structure specification unit 40 specifies an incorrect first tissue structure because the specific cross section specified by the cross section type specification unit 38 is incorrect. Other second learning models 34 include a second learning model 34 associated with a cross section type other than the specific cross section. That is, other second learning models 34 include a second learning model 34 corresponding to the true cross section type of the target image TI. Therefore, the tissue structure specification unit 40 can specify the second tissue structure, which is a correct tissue structure, on the basis of the outputs of other second learning models 34 corresponding to the true cross section type of the target image TI.

Next, a case will be considered where the tissue structure specification unit 40 specifies an incorrect first tissue structure because the specific cross section specified by the cross section type specification unit 38 is correct, but the initial second learning model 34 is not suitable for the target image TI. Other second learning models 34 include the second learning models 34 that are other than the initial second learning model 34 and are associated with the specific cross section. These second learning models 34 can include a second learning model 34 suitable for the target image TI. For example, even in a case where the brightness of the target image TI is too low for the initial second learning model 34 to predict the correct tissue structure, the tissue structure specification unit 40 can specify the second tissue structure, which is a correct tissue structure, on the basis of the outputs of other second learning models 34 as long as other second learning models 34 associated with the specific cross section perform preprocessing for correcting (increasing) the brightness.

The display controller 22 displays the second tissue structure information, which is information related to the second tissue structure specified by the tissue structure specification unit 40, on the display 24. FIG. 3 is a diagram illustrating a display example of second tissue structure information S2. In the present embodiment, the display controller 22 displays the second tissue structure information S2 on the display 24 together with the target image TI and the cross section information CS. The second tissue structure information S2 includes, for example, an icon indicating the position of the second tissue structure on the target image TI, or an image icon or characters indicating the label. In the example illustrated in FIG. 3, as the second tissue structure information S2, an icon indicating the position of the second tissue structure on the target image TI is displayed to be superimposed on the target image TI. Further, in addition to the icon, for example, characters indicating the label of the second tissue structure may be displayed. In addition, as illustrated in FIG. 3, the display controller 22 may display the second tissue structure information S2 together with the first tissue structure information S1. In this case, the icon or the like as the second tissue structure information S2 may have a shape or color different from that of the icon as the first tissue structure information S1.

Furthermore, before the second tissue structure information S2 is displayed on the display 24 or before the prediction process using the other second learning models 34, the display controller 22 may display a notification for the user's confirmation on the display 24. For example, a dialog or the like may be displayed in a pop-up manner.

In a case where the second tissue structure indicated by the second tissue structure information S2 is a correct tissue structure, the user can designate the second tissue structure (the designation also means notifying the ultrasound diagnostic apparatus 10 that the second tissue structure is a correct tissue structure) and proceed to the subsequent process, for example, a measurement process related to the second tissue structure.

As described above, according to the present embodiment, in a case where the first tissue structure is not correct, the second tissue structure is specified using other second learning models 34 including the second learning model 34 associated with the cross section type other than the specific cross section in response to the instruction from the user. Therefore, it is possible to improve the accuracy of specifying the tissue structure.

The following case will be considered: the tissue structure specification unit 40 specifies an incorrect first tissue structure because the specific cross section specified by the cross section type specification unit 38 is incorrect; and the tissue structure specification unit 40 specifies the second tissue structure on the basis of the outputs of other second learning models 34 associated with the cross section type other than the specific cross section, and the second tissue structure is a correct tissue structure. In this case, since the correct tissue structure can be specified on the basis of the outputs of other second learning models 34, other second learning models 34 are highly likely to be models specializing in predicting the tissue structure included in the ultrasound tomographic image having the true cross section type of the target image TI. That is, it can be said that the cross section type associated with other second learning models 34 is likely to be the true cross section type of the target image TI.

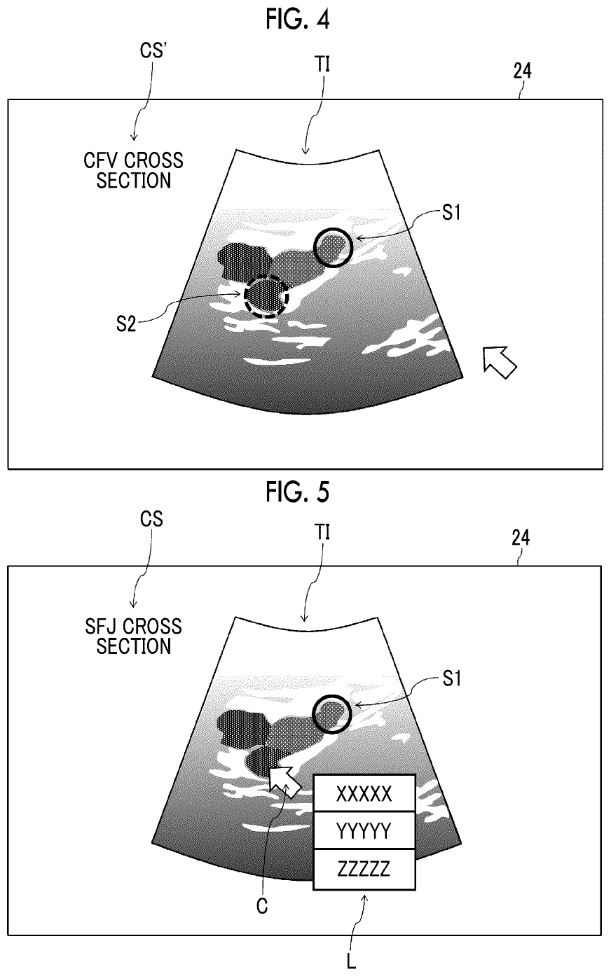

Therefore, in a case where the specific cross section specified by the cross section type specification unit 38 is different from the cross section type associated with other second learning models 34 that have contributed to the specification of the correct second tissue structure, the display controller 22 may display, on the display 24, corrected cross section information CS' indicating the cross section type corresponding to other second learning models 34 that have contributed to the specification of the second tissue structure, instead of the cross section information CS (see FIG. 3) indicating the specific cross section as illustrated in FIG. 4. Therefore, even in a case where the cross section type specification unit 38 specifies an incorrect specific cross section, it is possible to notify the user of the correct cross section type of the target image TI.

In the present embodiment, in a case where the first tissue structure is incorrect, the tissue structure specification unit 40 performs the process of specifying the second tissue structure using the other second learning models 34. Here, in some cases, a large number of second learning models 34 are stored in the memory 30. In this case, the number of other second learning models 34 to which the target image TI needs to be input may be large, and the amount of processing for specifying the second tissue structure may be enormous.

In consideration of the problem, a plurality of second learning models 34 may be grouped in advance, corresponding to each part of the subject. For example, a plurality of second learning models 34 may be classified into a plurality of groups such as a group corresponding to the abdomen and a group corresponding to the lower limb. In addition, the tissue structure specification unit 40 may input the target image TI to other second learning models 34 belonging to the same group as the initial second learning model 34 among a plurality of second learning models 34 other than the initial second learning model 34 and may not input the target image TI to the second learning model 34 belonging to a group different from the group to which the initial second learning model 34 belongs, in response to an instruction from the user who has checked the first tissue structure information S1. Then, the tissue structure specification unit 40 may specify the second tissue structure included in the target image TI on the basis of the prediction results of other second learning models 34 belonging to the same group as the initial second learning model 34.

This is based on the fact that, even in a case where the cross section type specification unit 38 specifies an incorrect specific cross section, there is a very low possibility that a part having a cross section different from the true cross section type of the target image TI will be determined as a specific part. For example, in a case where the true cross section type of the target image TI is the CFV cross section which is the cross section of the lower limb, the cross section type specification unit 38 is likely to specify the cross section of the target image TI as the SFJ cross section which is the cross section of the lower limb, but is less likely to specify the cross section of the target image TI as the cross section of the abdomen. In a case in which the cross section type specification unit 38 mistakenly specifies the cross section of the target image TI as the SFJ cross section which is the cross section of the lower limb, the initial second learning model 34 is associated with the SFJ cross section which is the cross section of the lower limb. Then, in this case, the tissue structure specification unit 40 inputs the target image TI to other second learning models 34 (including other second learning models 34 associated with the CFV cross section which is the true cross section type) corresponding to the lower limb in response to an instruction from the user. As a result, it is possible to specify the second tissue structure, which is a correct tissue structure, using only other second learning models 34 belonging to the group corresponding to the lower limb, without using the second learning models 34 belonging to the groups other than the group corresponding to the lower limb (in this case, the second tissue structure, which is a correct tissue structure, can be specified by other second learning models 34 associated with the CFV cross section). That is, it is possible to reduce the amount of processing for specifying the second tissue structure, without reducing the possibility of specifying the second tissue structure which is a correct tissue structure.

In the above-described embodiment, for example, the tissue structure specification unit 40 sets, as the second tissue structure, the tissue structure detected at a position closest to the position designated by the user. However, the tissue structure specification unit 40 may present a plurality of candidates for the second tissue structure to the user and allow the user to select the second tissue structure.

Specifically, first, the tissue structure specification unit 40 inputs the target image TI to each of a plurality of other second learning models 34 other than the initial second learning model 34 in response to an instruction from the user who has checked the first tissue structure information S1 (see FIG. 2). Then, the plurality of other second learning models 34 predict the positions and labels of a plurality of tissue structures.

Then, the tissue structure specification unit 40 calculates the order of prediction accuracy for each of the plurality of labels of the plurality of tissue structures predicted by the plurality of other second learning models 34. For example, the tissue structure specification unit 40 arranges the predicted tissue structures in order of proximity to the position designated by the user, arranges the labels of each tissue structures in the same order such that the labels do not overlap each other (for example, in a case where the same label is present, a label with a higher order is used, and the other labels are not used), and sets the arrangement order as the order of the prediction accuracy of the labels. Alternatively, a predetermined range having the position designated by the user as the center may be determined, and the descending order of the number of detections within the range may be used as the order of the prediction accuracy of the labels.

The display controller 22 displays the labels of the plurality of tissue structures predicted by each of the plurality of other second learning models 34 on the display 24 in a display aspect in which the calculated order of the prediction accuracy is represented. FIG. 5 is a diagram illustrating a display example of the labels of the plurality of tissue structures. In the example illustrated in FIG. 5, the labels of the plurality of predicted tissue structures are displayed in the form of a label list L. In the label list L, a plurality of labels are displayed to be arranged in the vertical direction, the label displayed at the top has the highest prediction accuracy, and the label displayed on the lower side has the lower prediction accuracy. Of course, the label list L is only an example, and the display controller 22 may display the plurality of labels in any display aspect as long as the order of the prediction accuracy is represented.

The user checks the plurality of labels displayed in the display aspect, in which the order of the prediction accuracy is represented, and selects one label with reference to the prediction accuracy.

The tissue structure specification unit 40 specifies, as the second tissue structure, a tissue structure related to the label selected by the user among the plurality of tissue structures predicted by each of the plurality of other second learning models 34. In addition, in a case where there are a plurality of tissue structures related to the label selected by the user, the tissue structure specification unit 40 selects one tissue structure from the plurality of tissue structures related to the selected label and sets the selected tissue structure as the second tissue structure. For example, the tissue structure specification unit 40 sets, as the second tissue structure, a tissue structure at a position closest to the position designated by the user among the plurality of tissue structures related to the selected label.

The process of the image quality adjustment unit 20 will be described in detail. The image quality adjustment information 36 stored in the memory 30 is information in which the cross section type of the ultrasound tomographic image is associated with image quality adjustment parameters for adjusting the quality of the ultrasound tomographic image of the cross section type. The image quality adjustment unit 20 adjusts the quality of the target image TI on the basis of the cross section type of the target image TI and the image quality adjustment information 36.

In particular, the following case will be considered: the tissue structure specification unit 40 specifies an incorrect first tissue structure because the specific cross section specified by the cross section type specification unit 38 is incorrect; and the tissue structure specification unit 40 specifies a second tissue structure on the basis of the outputs of other second learning models 34 associated with the cross section type other than the specific cross section, and the second tissue structure is a correct tissue structure. That is, a case is considered where the specific cross section is different from the cross section type associated with other second learning models 34 that have contributed to the specification of the second tissue structure.

In this case, the image quality adjustment unit 20 may adjust the quality of the target image TI using the image quality adjustment parameters associated with the cross section type corresponding to other second learning models 34 that have contributed to the specification of the second tissue structure. This makes it possible to adjust the quality of the target image TI using the image quality adjustment parameters suitable for the true cross section type of the target image TI.

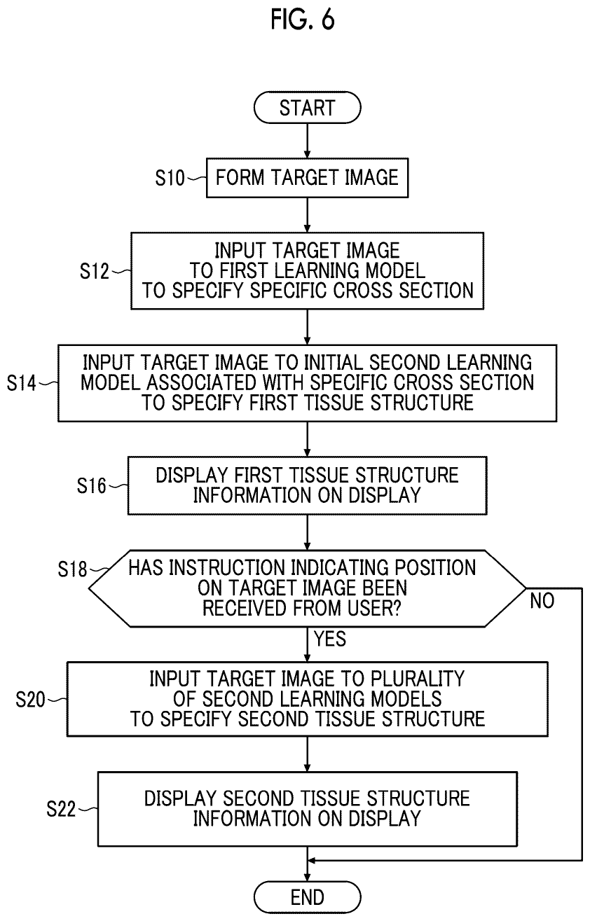

The outline of the configuration of the ultrasound diagnostic apparatus 10 according to the present embodiment is as described above. Hereinafter, a flow of a basic process of the ultrasound diagnostic apparatus 10 will be described with reference to a flowchart illustrated in FIG. 6. In addition, it is assumed that the first learning model 32 and the second learning model 34 have been trained at the starting time of the flowchart illustrated in FIG. 6.

In Step S10, the image forming unit 18 forms the target image TI, which is an ultrasound tomographic image, on the basis of the received beam signal subjected to the signal processing in the signal processing unit 16.

In Step S12, the cross section type specification unit 38 inputs the target image TI formed in Step S10 to the first learning model 32 and specifies a specific cross section, which is the cross section type of the target image TI, on the basis of the prediction result of the first learning model 32 for the target image TI.

In Step S14, the tissue structure specification unit 40 inputs the target image TI to the initial second learning model 34 associated with the specific cross section specified in Step S12 and specifies the first tissue structure included in the target image TI on the basis of the prediction result of the initial second learning model 34 for the target image TI.

In Step S16, the display controller 22 displays the first tissue structure information related to the first tissue structure specified in Step S14 on the display 24.

In Step S18, the tissue structure specification unit 40 determines whether or not an instruction indicating a position on the target image TI has been received from the user who has checked the first tissue structure information displayed on the display 24 in Step S16. In a case where the instruction has not been received, it means that the first tissue structure is not incorrect (is the tissue structure expected by the user). Therefore, the process in the flowchart is ended. Then, the user proceeds to the measurement process or the like related to the first tissue structure. In a case where the instruction has been received, the process proceeds to Step S20.

In Step S20, the tissue structure specification unit 40 inputs the target image TI to other second learning models 34 other than the initial second learning model 34 and specifies the second tissue structure included in the target image TI on the basis of the prediction results of other second learning models 34 for the target image TI.

In Step S22, the display controller 22 displays the second tissue structure information related to the second tissue structure specified in Step S20 on the display 24.

Then, in a case where the second tissue structure is a correct tissue structure, the user selects the second tissue structure and then proceeds to the measurement process or the like related to the second tissue structure.

The ultrasound tomographic image processing apparatus according to the present disclosure has been described above. However, the ultrasound tomographic image processing apparatus according to the present disclosure is not limited to the above-described embodiment, and various changes can be made without departing from the gist of the present disclosure.

For example, in the above-described embodiment, the ultrasound tomographic image processing apparatus is the ultrasound diagnostic apparatus 10. However, the ultrasound tomographic image processing apparatus is not limited to the ultrasound diagnostic apparatus 10. For example, the ultrasound tomographic image processing apparatus may be a personal computer (PC), a server, or the like. In this case, the PC or the server as the ultrasound tomographic image processing apparatus has the display controller 22, the display 24, the memory 30 in which the trained first learning model 32 and the trained second learning model 34 are stored, the cross section type specification unit 38, and the tissue structure specification unit 40. The PC or the server as the ultrasound tomographic image processing apparatus acquires the target image TI from the ultrasound diagnostic apparatus and performs the process according to the above-described embodiment on the acquired target image TI.

In addition, in the above-described embodiment, the first learning model 32 and the second learning model 34 are stored in the memory 30 of the ultrasound tomographic image processing apparatus. However, the first learning model 32 and the second learning model 34 may be stored in an apparatus different from the ultrasound tomographic image processing apparatus as long as the first learning model 32 and the second learning model 34 can be accessed from the ultrasound tomographic image processing apparatus.

What is claimed is:

1. An ultrasound tomographic image processing apparatus capable of accessing a first learning model that has been trained to predict a cross section type of an input ultrasound tomographic image and to output the cross section type and a plurality of second learning models each of which is associated with each cross section type of ultrasound tomographic images and has been trained to predict a tissue structure included in the ultrasound tomographic image of a corresponding cross section type and to output the tissue structure, the ultrasound tomographic image processing apparatus comprising:
a cross section type specification unit that inputs a target image, which is an ultrasound tomographic image to be processed, to the first learning model to specify a specific cross section which is a cross section type of the target image;
a tissue structure specification unit that inputs the target image to an initial second learning model, which is the second learning model associated with the specific cross section, to specify a first tissue structure included in the target image; and
a display controller that displays first tissue structure information, which is information related to the first tissue structure, on a display unit,
wherein the tissue structure specification unit inputs the target image to the second learning models, which are other than the initial second learning model and include the second learning model associated with a cross section type other than the specific cross section, in response to an instruction indicating a position on the target image from a user who has checked the first tissue structure information and specifies a second tissue structure included in a vicinity of the position indicated by the instruction from the user in the target image on the basis of prediction results of the second learning models.

2. The ultrasound tomographic image processing apparatus according to claim 1,
wherein the display controller displays cross section information indicating the specific cross section on the display unit, and
in a case where the specific cross section is different from a cross section type associated with the second learning model that has contributed to the specification of the second tissue structure, the display controller displays corrected cross section information indicating the cross section type corresponding to the second learning model that has contributed to the specification of the second tissue structure on the display unit, instead of the cross section information indicating the specific cross section.

3. The ultrasound tomographic image processing apparatus according to claim 1,
wherein the plurality of second learning models are grouped corresponding to each part of a subject, and
the tissue structure specification unit inputs the target image to the second learning model belonging to the same group as the initial second learning model among the plurality of second learning models other than the initial second learning model in response to an instruction from the user who has checked the first tissue structure information and further specifies the second tissue structure included in the target image on the basis of a prediction result of the second learning model.

4. The ultrasound tomographic image processing apparatus according to claim 1,
wherein the tissue structure specification unit inputs the target image to each of a plurality of the second learning models other than the initial second learning model in response to an instruction from the user who has checked the first tissue structure information and calculates an order of prediction accuracy for each of labels of a plurality of tissue structures predicted by the plurality of second learning models,
the display controller displays the labels of the plurality of tissue structures predicted by the plurality of second learning models on the display unit in a display mode in which the order of the prediction accuracy is represented, and
the tissue structure specification unit specifies, as the second tissue structure, a tissue structure related to a label selected by the user among the plurality of tissue structures predicted by the plurality of second learning models.

5. The ultrasound tomographic image processing apparatus according to claim 1, further comprising:
an image quality adjustment unit that adjusts quality of the ultrasound tomographic image on the basis of image quality adjustment information in which the cross section type of the ultrasound tomographic image is associated with an image quality adjustment parameter for adjusting the quality of the ultrasound tomographic image of the cross section type and that, in a case where the specific cross section is different from the cross section type associated with the second learning model which has contributed to the specification of the second tissue structure, adjusts quality of the target image using the image quality adjustment parameter associated with the cross section type corresponding to the second learning model which has contributed to the specification of the second tissue structure.

6. A non-transitory computer-readable storage medium storing an ultrasound tomographic image processing program causing a computer, which is capable of accessing a first learning model that has been trained to predict a cross section type of an input ultrasound tomographic image and to output the cross section type and a plurality of second learning models each of which is associated with each cross section type of ultrasound tomographic images and has been trained to predict a tissue structure included in an ultrasound tomographic image of a corresponding cross section type and to output the tissue structure, to function as:
a cross section type specification unit that inputs a target image, which is an ultrasound tomographic image to be processed, to the first learning model to specify a specific cross section which is a cross section type of the target image;
a tissue structure specification unit that inputs the target image to an initial second learning model, which is the second learning model associated with the specific cross section, to specify a first tissue structure included in the target image; and
a display controller that displays first tissue structure information, which is information related to the first tissue structure, on a display unit,
wherein the tissue structure specification unit inputs the target image to the second learning models, which are other than the initial second learning model and include the second learning model associated with a cross section type other than the specific cross section, in response to an instruction indicating a position on the target image from a user who has checked the first tissue structure information and specifies a second tissue structure included in a vicinity of the position indicated by the instruction from the user in the target image on the basis of prediction results of the second learning models.
